(19) United States
(12) Patent Application Publication
(10) Pub. No.: US 2024/0201169 A1
(43) Pub. Date: Jun. 20, 2024

(54) METHODS OF THREE-DIMENSIONAL TUMOR SPHEROID MICROARRAY FOR HIGH-THROUGHPUT, HIGH-CONTENT IMMUNE CELL-MEDIATED CYTOTOXICITY

(71) Applicants: Seok-Joon KWON, Niskayuna, NY (US); Jonathan Seth DORDICK, Schenectady, NY (US); Sneha GOPAL, Troy, NY (US); Bosung KU, Yongin-si (KR)

(72) Inventors: Seok-Joon KWON, Niskayuna, NY (US); Jonathan Seth DORDICK, Schenectady, NY (US); Sneha GOPAL, Troy, NY (US); Bosung KU, Yongin-si (KR)

(73) Assignee: RENSSELAER POLYTECHNIC INSTITUTE, Troy, NY (US)

(21) Appl. No.: 18/276,349

(22) PCT Filed: Feb. 8, 2022
(86) PCT No.: PCT/US2022/015568

§ 371 Date: Aug. 8, 2023

Related U.S. Application Data
(60) Provisional application No. 63/147,189, filed on Feb. 8, 2021.

(51) Int. Cl. G01N 33/50; C12M 1/32; C12M 3/00; C12N 5/0783; C12N 5/09

(52) U.S. Cl. CPC G01N 33/5011; C12M 23/12; C12M 23/44; C12N 5/0646; C12N 5/0693; C12N 2503/00; C12N 2533/52

(57) ABSTRACT

A chip system is provided that includes a micropillar chip and a corresponding microwell chip. The micropillars are coated with an anchoring layer, e.g., polydopamine/polylysine/fibronectin, which anchors an amount of a hydrogel composition to the micropillars, the composition including a matrix and a concentration of cancer cells, such as those from a particular patient. Upon gelation, the composition forms a three-dimensional structure with a high cancer cell density mimicking a tumor microenvironment, e.g., allows culturing of the cancer cells in hypoxic conditions representative of the conditions at the patient's tumor. The 3D structures of composition on the micropillars are then stamped into corresponding microwells including media composed of a variety of treatments for testing against the cancer cells. The media can include immune cells, enabling co-culture of the cancer cells with the immune cells to test for immune cell-mediated cytotoxicity, as well as combinations of drug and antibody based therapies.

### CROSS REFERENCE TO RELATED APPLICATION(S)

This application is a national stage filing of International Patent Application No. PCT/US2022/015568, filed Feb. 8, 2022, which claims the benefit of U.S. Provisional Application No. 63/147,189, filed Feb. 8, 2021, which is incorporated by reference as if disclosed herein in its entirety.

### BACKGROUND

Cancer immunotherapy has emerged as an effective regimen alone or in combination with other treatments, such as surgery, chemotherapy, and radiation therapy. With the rapid increase in our understanding of the immune system, an increasing number of small molecules, peptides, recombinant antibodies, vaccines, and cellular therapeutics have been applied to manipulate the immune response for cancer treatment. These immunotherapies have provided significant benefits in the fight against cancer, especially the application of immune checkpoint inhibitors and cell-based therapies. Unfortunately, solid tumors, such as pancreatic or breast cancer, are often resistant to immunotherapy. A major reason for this is the immunosuppressive effect of the tumor microenvironment (TME) that includes abundant stroma, protumor macrophages and regulatory T cells that prevent proliferation of other effector T cells. Together with the tumor cells, this complex ecosystem can lead to immune escape and immunotherapy resistance.

Cancer immunotherapy alone or in combination with antibodies and small molecule chemotherapeutics can, in principle, overcome immune-driven immunosuppression, with combination treatment being currently regarded as a promising approach. In particular the use of natural killer (NK) cells has been investigated for their potential to induce cytotoxicity in cancer cells. However, given the large number of approved and candidate drugs, it is not feasible to compare all immunotherapy agents in a clinical setting. Therefore, various experimental ex vivo models have been developed to study combination treatment, including classical 2D cell culture models, tumor spheroids and organoids, and patient-derived xenografts. Among these models, conventional 2D cell culture cannot effectively recapitulate the complexity of cellular interactions and mimic the in vivo TME. Furthermore, patient-derived xenografts suffer from limited scalability especially for screening multiple combination therapies. Tumor spheroid and organoid models have been adapted for high throughput therapeutic drug screening. Novel platform designs that incorporate tumor-immune cell co-cultures in contextually relevant 3D microenvironments can prove useful for studying antibody-dependent cellular cytotoxicity (ADCC), leading to personalized therapy regimens for patients.

To effectively identify successful combination therapies, it is critical to evaluate the cytotoxic efficacies of effector cells, such as NK and T cells against target cancer cells. A ⁵¹Cr release assay has long been the most widely used method for quantification of ADCC by measuring the radioactivity of ⁵¹Cr released from dead cells. However, the reproducibility, sensitivity, and specificity of the ⁵¹Cr release assay are not adequate because of spontaneous release of ⁵¹Cr-labelled target cells. In addition, the ⁵¹Cr release assay is unsafe for untrained researchers to handle because of its radioactivity. Non-radioactive probes, such as Calcein-AM or lanthanide chelates have been used as alternatives to the ⁵¹Cr-release assay wherein cells are prelabeled with the dyes prior to treatment with effector cells and cytotoxicity is measured via release of the dye. Nonetheless, these methods also show high levels of spontaneous release of the probes, and target cell line-dependent labeling variability result from the activity of intracellular esterases. Another method for evaluating ADCC is flow cytometry after labeling target and effector cells with different fluorescent probes and staining with cell viability dyes. These methods specifically quantify target cell death or disappearance, which provide more reproducible and sensitive data. However, flow cytometry often shows relatively high sample-to-sample variation, and can require long processing times depending on the number of samples.

### SUMMARY

Aspects of the present disclosure are directed to a method of performing cancer-immune cell co-culture. In some embodiments, the method includes providing a micropillar chip with corresponding microwell chip. In some embodiments, the method includes applying an anchoring layer to the micropillar chip. In some embodiments, the method includes preparing a composition including a plurality of cancer cells, e.g., from a cancer cell line, primary cells from a tumor, or combinations thereof. In some embodiments, the method includes preparing a composition including a matrix. In some embodiments, the method includes preparing a composition including a matrix and a plurality of cancer cells. In some embodiments, the method includes spotting an amount of the composition on the anchoring layer of a plurality of micropillars on the micropillar chip. In some embodiments, the method includes incubating the composition on the micropillar chip. In some embodiments, the method includes applying a medium to microwells of the microwell chip, the medium including a cell culture including a concentration of immune cells. In some embodiments, the method includes immersing the plurality of micropillars in the medium to facilitate co-culture of the cancer cells and the immune cells. In some embodiments, the method includes identifying a cytotoxic effect of the medium on the cancer cells.

In some embodiments, the anchoring layer includes polydopamine, polylysine, fibronectin, laminin, collagen, or combinations thereof. In some embodiments, the composition is a hydrogel. In some embodiments, the matrix includes Matrigel, alginate, collagen, peptides, or combinations thereof. In some embodiments, the composition further comprises one or more extracellular matrix proteins. In some embodiments, the extracellular matrix proteins include fibronectin, laminin, or combinations thereof. In some embodiments, the amount of the composition is less than about 1 μL. In some embodiments, the amount of composition is between about 60 nL and about 500 nL. In some embodiments, the composition has a cell concentration between about 1×10⁵ cells/mL and about 1×10⁹ cells/mL. In some embodiments, the immune cells include lymphocytes, monocytes, macrophages, peripheral blood mononuclear cells, dendritic cells, or combinations thereof. In some embodiments, the medium further includes one or more treatments, the treatments including a concentration of antibodies, a concentration of therapeutic small molecules, or combinations thereof.

Aspects of the present disclosure are directed to a method of evaluating cancer cell cytotoxicity. In some embodiments, the method includes providing a chip system including a micropillar chip and a microwell chip, the micropillar chip including a plurality of micropillars configured to fit within a corresponding plurality of microwells on the microwell chip. In some embodiments, the method includes applying an anchoring layer to the plurality of micropillars. In some embodiments, the anchoring layer includes polydopamine, polylysine, fibronectin, laminin, collagen, or combinations thereof. In some embodiments, the method includes preparing a composition including a matrix and a plurality of cancer cells, e.g., from a cancer cell line, primary cells from a tumor, or combinations thereof. In some embodiments, the method includes spotting an amount of the composition on the anchoring layer on a plurality of the micropillars. In some embodiments, the method includes incubating the composition on the micropillar chip to form sphereoids of composition. In some embodiments, the method includes applying growth medium to the microwells of the microwell chip. In some embodiments, the method includes immersing the spheroids in the growth medium. In some embodiments, the method includes applying one or more treatments against the cancer cells to the microwells. In some embodiments, the method includes identifying the effect of the one or more treatments on the plurality of cells. In some embodiments, the one or more treatments include a concentration of natural killer (NK) cells, a concentration of antibodies, a concentration of therapeutic small molecules, or combinations thereof.

In some embodiments, the composition is a hydrogel. In some embodiments, the matrix includes Matrigel, alginate, collagen, peptides, or combinations thereof. In some embodiments, the composition further comprises one or more extracellular matrix proteins, the extracellular matrix proteins including fibronectin, laminin, or combinations thereof. In some embodiments, the amount of composition is between about 60 nL and about 500 nL. In some embodiments, the composition has a cell concentration between about 1×10⁵ cells/mL and about 1×10⁹ cells/mL.

Aspects of the present disclosure are directed to a chip system. In some embodiments, the chip system includes a micropillar chip and a microwell chip, the micropillar chip including a plurality of micropillars configured to fit within corresponding microwells on the microwell chip. In some embodiments, the chip system includes an anchoring layer on at least a portion of the micropillars. In some embodiments, the anchoring layer includes polydopamine, polylysine, fibronectin, laminin, collagen, or combinations thereof. In some embodiments, the chip system includes an amount of a hydrogel composition on the anchoring layer. In some embodiments, the hydrogel composition includes a matrix including Matrigel, alginate, collagen, peptides, or combinations thereof. In some embodiments, the hydrogel composition includes a concentration of cancer cells, e.g., a cancer cell line, primary cells from a tumor, or combinations thereof, between about 1×10⁵ cells/mL and about 1×10⁹ cells/mL composition. In some embodiments, the hydrogel composition includes a matrix and a concentration of cancer cells. In some embodiments, the amount of the hydrogel composition is between about 60 nL and about 500 nL. In some embodiments, the hydrogel composition further comprises one or more extracellular matrix proteins.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a chart of a method of performing cytotoxicity studies of cancer cell lines according to some embodiments of the present disclosure;

FIG. 2 is a chart of a method of performing cytotoxicity studies of cancer cell lines according to some embodiments of the present disclosure;

FIG. 3 is a schematic representation of a chip system according to some embodiments of the present disclosure;

FIGS. 4A-4B portray results demonstrating NK92-CD16 cell-mediated killing of MiPaCa2 cells using a 384-pillar/well plate sandwich system using both upward (upside-down) and downward micropillars into microwells;

FIGS. 5A-5C portray results demonstrating antibody-dependent cell-mediated cytotoxicity (ADCC) using a 2D culture-based 384-pillar/well plate sandwich platform;

FIGS. 6A-6D portray results demonstrating NK92-CD16 cell-mediated killing against 3D MCF-7 cell aggregates in Matrigel;

FIGS. 7A-7B portray generation of 3D tumor spheroid micropillar arrays according to some embodiments of the present disclosure;

FIGS. 8A-8B portray results showing cytotoxicity of cancer cells at different effector to target ratios as demonstrated using systems according to some embodiments of the present disclosure; and

FIGS. 9A-9C portray results showing cytotoxicity of cancer cells in the presence of Trastuzumab and Atezolizumab as demonstrated using systems according to some embodiments of the present disclosure.

### DETAILED DESCRIPTION

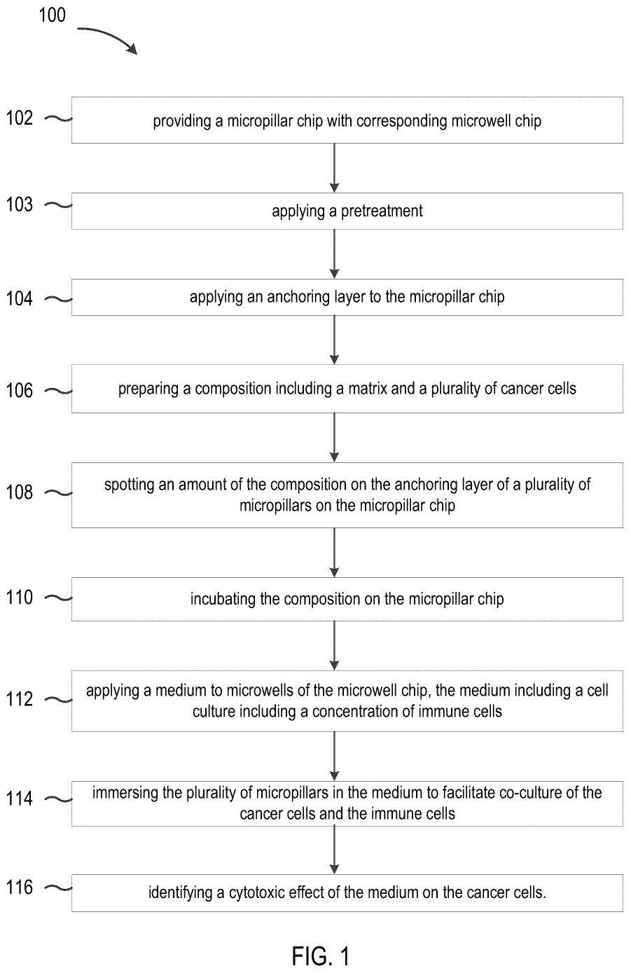

Referring now to FIG. 1, some embodiments of the present disclosure are directed to a method 100 of performing cytotoxicity studies of cancer cells, e.g., from cancer cell lines, primary cells from a tumor, or combinations thereof. In some embodiments, method 100 evaluates the cytotoxicity of a medium for a given sample of cancer cells, e.g., from a cancer cell line, primary cells from a tumor, or combinations thereof, wherein the medium includes one or more treatments. In some embodiments, medium includes immune cells, and thus enables a method 100 that includes co-culture of immune cells with cancer cells. In some embodiments, method 100 includes culture of cancer cells in an environment that mimics a tumor microenvironment, and thus allows for the testing of treatments against the sample of cancer cells in an environment more representative of that found in a patient. In some embodiments, method 100 includes screening combinations of drugs, antibodies, and immune cells to identify effective combinations of these components against the sample of cancer cells. In some embodiments, the treatments include immune cell-mediated cytotoxicity, and antibody dependent cell-mediated cytotoxicity, drug/drug combinations, drug/antibody combinations, or antibody/antibody combinations, all with or without immune cell mediation. In some embodiments, the sample of cancer cells is directly from or derived from a particular patient, and thus the testing of personalized cancer treatments is enabled.

Still referring to FIG. 1, in some embodiments, at 102, a chip system is provided. In some embodiments, the chip system is composed of any suitable material or combination of materials for fixing and culturing tumor cells, immune cells, etc. In some embodiments, the chip system includes one or more polymers, e.g., polystyrene. In some embodiments, the chip system is any suitable size for facilitating the culture of cancer cells in an environment that mimics a tumor microenvironment consistent with the embodiments described herein, as will be discussed in greater detail below.

In some embodiments, the chip system includes a micropillar chip and a microwell chip. In some embodiments, the micropillar chip includes a plurality of micropillars that are configured to fit into corresponding microwells on the microwell chip. The micropillars and the microwells can be of any suitable size and shape for facilitating the culture of cancer cells in an environment that mimics a tumor microenvironment consistent with the embodiments described herein. Further, the micropillars and microwells are sized and shaped such that a micropillar can be accepted into the microwell while allowing a space between the two structures to receive a volume of liquid. In some embodiments, the space has a volume and shape sufficiently small to limit or prevent flow of the volume of liquid within the space via surface tension, even during application of external forces to or inversion of the chip system. In an exemplary embodiment, the diameter of each pillar and well is 1 mm and 1.9 mm, respectively. While exemplary embodiments of the present disclosure described below describe chip systems including 330 or 384 micropillars/microwells, the present disclosure is not limited in this regard, as the chips may have greater than 50, 100, 200, 300, 400, etc. micropillars and associated microwells without departing from the embodiments of the present disclosure.

In some embodiments, at 103, one or more components of the chip system undergoes one or more pretreatments. In some embodiments, the chip system is UV-treated, e.g., using a 96 W transilluminator (Syngene GVM-30) for 4 h, to enhance surface hydrophilicity. In some embodiments, pretreatment 103 includes one or more plasma treatments.

At 104, an anchoring layer is applied to the micropillar chip. In some embodiments, the anchoring layer includes polydopamine, polylysine, fibronectin, laminin, collagen, or combinations thereof. In some embodiments, an anchoring layer is applied to a plurality of the micropillars on the micropillar chip. In some embodiments, an anchoring layer is applied to all of the micropillars on the micropillar chip. In some embodiments, when the anchoring layer is applied to a micropillar, the layer covers at least a portion of the micropillar, e.g., the tip of the micropillar, the tip and a portion of the sides of the micropillar, etc. In some embodiments, when the anchoring layer is applied to a micropillar, the layer covers the entire surface of the micropillar.

At 106, a composition is prepared. In some embodiments, the composition is a hydrogel. In some embodiments, the composition includes a matrix. In some embodiments, the composition includes a plurality of cancer cells. In some embodiments, the composition includes a matrix and a plurality of cancer cells. In some embodiments, the plurality of cancer cells is from a cancer cell line, primary cells from a tumor, or combinations thereof. In some embodiments, the matrix includes Matrigel, alginate, collagen, peptides, or combinations thereof. In some embodiments, one or more cancer cell lines and/or primary cells from a tumor are cultivated. In some embodiments, the cancer cells are from a known cancer cell line, e.g., MiaPaCa-2 pancreatic cancer cells, MCF7 and/or MDA-MB-231 breast cancer cells, etc. In some embodiments, the cancer cells are from a patient's own cancer cells. In some embodiments, the composition has a cell concentration between about 1×10⁵ cells/mL and about 1×10⁹ cells/mL. In some embodiments, the composition has a cell concentration between about 1×10⁷ cells/mL. In some embodiments, the cancer cells are detached and mixed with one or more components, e.g., the matrix component, to form the composition. In some embodiments, the composition includes one or more extracellular matrix proteins. In some embodiments, the extracellular matrix proteins include fibronectin, laminin, or combinations thereof.

At 108, an amount of the composition is spotted on the micropillars. In some embodiments, an amount of the composition is spotted 108 on at least a subset of the micropillars. In some embodiments, an amount of the composition is spotted 108 on all of the micropillars. In some embodiments, the amount spotted 108 on the micropillars is substantially uniform. In some embodiments, the amount of the composition spotted 108 is less than about 1 μL. In some embodiments, the amount of the composition spotted 108 is between about 60 nL and about 500 nL. In some embodiments, about 50, 100, 150, 200, 250, 300, 350, 400, 450, 500 nL, or combinations thereof, of composition is spotted 108 on the micropillars. In some embodiments, the composition is spotted 108 on at least some of the micropillars using an ASFA™ spotter.

In some embodiments, when the composition is spotted 108 on a micropillar, it is positioned on a surface thereof, e.g., on the tip of the micropillar. In some embodiments, when the composition is spotted 108 on a micropillar, it is positioned on the anchoring layer. The anchoring coating on the micropillar surface allows for covalent coupling between coating and proteins in the composition, e.g., with Matrigel. In some embodiments, when the composition is spotted 108 on a micropillar, the composition forms a hemispherical spot. In some embodiments, when the composition is spotted 108 on a micropillar, the composition forms a planar layer.

At 110, the composition on the micropillar chip is incubated. In some embodiments, the micropillar chip is incubated in any environment and at any given temperature for any given duration to facilitate gelation of the composition, e.g., in a humidity chamber for 15 min at 37° C. In some embodiments, the micropillar chip is incubated upside-down. As discussed above, in some embodiments, after incubation, the micropillar chips have generally planar masses of composition ready for testing against, e.g., a desired treatment or series of desired treatments. In some embodiments, the micropillar chips have generally spherical and/or hemi-spherical masses of composition, referred to herein also as “sphereoids,” ready for testing against. Spheroids can be highly resistant to drug exposure. In addition, cells within the sphereoids can have varying responses to the drug depending upon where they are located within the spheroid. For example, cells at the center of a spheroid have been found to be more than 90% viable even after 3 days of drug exposure, presumably because of very low or no cell growth deep within the spheroid structure.

The sphereoids are advantageous in that they mimic the extracellular matrix typically experienced by cancer cells in vivo, e.g., in a patient's tumor, and further include a high cancer cell density. As a result, the sphereoids can accurately mimic the hypoxic environment of the tumor microenvironment. As certain cancer cell lines have been shown to be increasingly drug resistant in a hypoxic environment, as might be found within a patient, the systems and methods of the present disclosure enable more accurate and higher throughput testing of interventions against those cell lines. In embodiments of the present disclosure, EC₅₀ values were measured for the paclitaxel concentration at which NK92-CD16 cells caused apoptosis of the cells in the presence of drugs and antibodies over a shorter 24 h time period. Since the platforms of the instant disclosure can be used to generate spheroids with hypoxic conditions in the interior of the sphereoids, high EC₅₀ values are to be expected.

At 112, a medium is applied to microwells of the microwell chip, e.g., those microwells corresponding to micropillars having composition spotted thereon. In some embodiments, the wells include about 3-4 μL of medium. In some embodiments, the medium includes growth medium, e.g., fresh Dulbecco's Modified Eagle Medium (DMEM). In some embodiments, the wells include about 3.6 μL of medium. In some embodiments, the medium includes one or more components desired for testing against the cancer cells. In some embodiments, the medium includes immune cells from one or more immune cell lines. In some embodiments, the immune cells include lymphocytes, monocytes, macrophages, peripheral blood mononuclear cells, dendritic cells, or combinations thereof. In some embodiments, the medium includes one or more treatments, e.g., for testing against the cancer cells in the sphereoids. In some embodiments, the treatments include a concentration of natural killer (NK) cells, a concentration of antibodies, a concentration of therapeutic small molecules, or combinations thereof. While the present disclosure details various exemplary embodiments with representative NK cell lines, antibodies, and small molecules, the present disclosure is not intended to be limited in this regard, as the platforms detailed herein allow high-throughput screening of any cell line, antibody, small molecule, or combination thereof to test their efficacy against the cancer cells.

At 114, the plurality of micropillars is immersed in the medium. The micropillars and the microwells of the chip system are sized and shaped such that when the micropillars are immersed in the medium, the medium is displaced and covers or substantially covers the composition spotted on the micropillar without forcing medium out of the microwell. As discussed above, the dimensions of the micropillars and the microwells are generally similar such that surface tension limits flow of the medium positioned between the micropillars and the walls of the microwells, maintaining contact between the composition and the medium even when the chip system subjected to external forces, such as jostling or inversion. In exemplary embodiments where the medium includes immune cells, immersing 114 facilitates co-culture of the cancer cells and the immune cells. Further, immersing 114 positions the composition with the cancer cells included therein in proximity with immune cell/drug/antibody combinations, so that the efficacy of these combinations against the cancer cells can be identified. In some exemplary embodiments, the micropillar chip, stamped onto the microwell chip, is further incubated, e.g., upside down for at least 24 h. In some exemplary embodiments, the chips are incubated for a period of about 3 days. In some embodiments, the micropillar chip is then washed, e.g., with 8 mL DPBS added to a 4-well rectangular dish. At 116, the effect of the medium on the cancer cells is identified. In some embodiments, the effect is increased cytotoxicity towards the cancer cells. The effects can be qualitatively and/or quantitatively identified via any suitable process or system, e.g., via staining, imaging, protein expression quantification, etc.

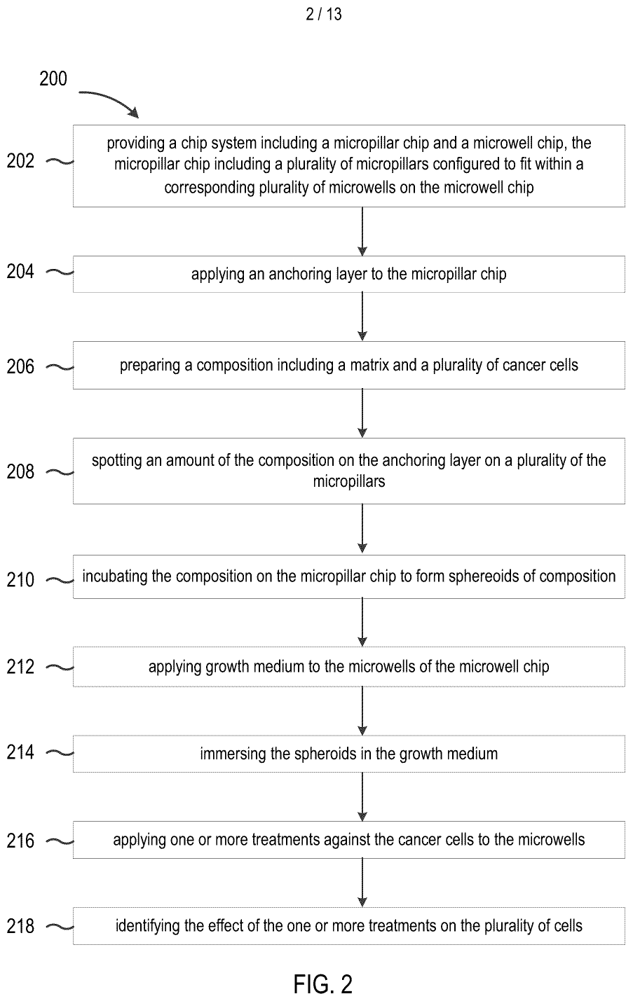

Referring now to FIG. 2, some embodiments of the present disclosure are directed to a method 200 of evaluating cancer cell cytotoxicity. In some embodiments, at 202, a chip system including a micropillar chip and a microwell chip is provided. As discussed above, the micropillar chip includes a plurality of micropillars configured to fit within a corresponding plurality of microwells on the microwell chip. At 204, an anchoring layer is applied to the plurality of micropillars. In some embodiments, the anchoring layer includes polydopamine, polylysine, fibronectin, laminin, collagen, or combinations thereof. At 206, a composition including a matrix and a plurality of cancer cells is prepared. In some embodiments, the plurality of cancer cells is from a cancer cell line, primary cells from a tumor, or combinations thereof. In some embodiments, the composition is a hydrogel. In some embodiments, the matrix includes Matrigel, alginate, collagen, peptides, or combinations thereof. In some embodiments, the composition further comprises one or more extracellular matrix proteins, the extracellular matrix proteins including fibronectin, laminin, or combinations thereof. In some embodiments, the composition has a cell concentration between about 1×10⁵ cells/mL and about 1×10⁹ cells/mL. In some embodiments, the composition has a cell concentration of about 1×10⁷ cells/mL. At 208, an amount of the composition is spotted on the anchoring layer on a plurality of the micropillars. In some embodiments, the amount of composition is between about 60 nL and about 500 nL. At 210, the composition is incubated on the micropillar chip. As discussed above, in some embodiments, incubation 210 produces generally planar masses of composition on the micropillars. In some embodiments, incubation 210 produces sphereoids of composition on the micropillars.

At 212, growth medium is applied to the microwells of the microwell chip. At 214, the spheroids are immersed in the growth medium. At 216, one or more treatments against the plurality of cancer cells are applied to the microwells. In some embodiments, the one or more treatments include a concentration of NK cells, a concentration of antibodies, a concentration of therapeutic small molecules, or combinations thereof. At 216, the effect of the one or more treatments on the plurality of cells is identified.

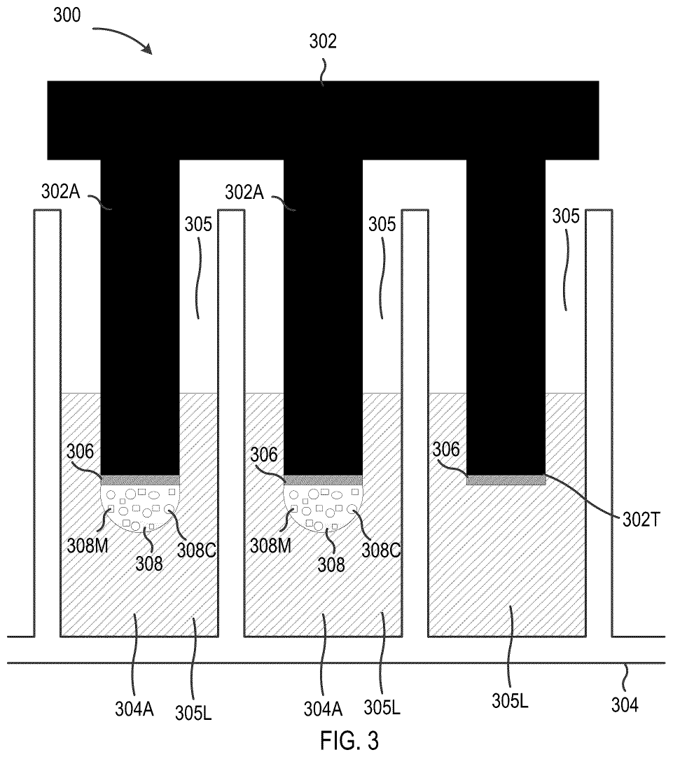

Referring now to FIG. 3, some embodiments of the present disclosure are directed to a chip system 300. In some embodiments, chip system 300 includes a micropillar chip 302 and a microwell chip 304. In some embodiments, micropillar chip 302 includes a plurality of micropillars 302A configured to fit within corresponding microwells 304A on microwell chip 304. In the exemplary embodiment shown in FIG. 3, micropillars 302A and microwells 304A are generally cylindrically shaped. Further, the micropillars and microwells are sized and shaped such that a micropillar can be accepted into the microwell while allowing a space 305 between micropillars 302A and microwells 304A to receive a volume of liquid 305L, such as the media discussed above with respect to method 100 and method 200. As discussed above, micropillars 302A and microwells 304A can be of any suitable size and shape for facilitating the culture of cancer cells in an environment that mimics a tumor microenvironment consistent with the embodiments described herein. Further, space 305 has a volume and shape sufficiently small to limit or prevent flow of liquid 305L via surface tension. In an exemplary embodiment, the diameter of each pillar and well is 1 mm and 1.9 mm, respectively. In some embodiments, chip system 300 has greater than about 50, 100, 200, 300, 400, etc. micropillars 302A and associated microwells 304A.

In some embodiments, an anchoring layer 306 is positioned on at least a portion of micropillars 302A, e.g., on the top surface 302T. In some embodiments, an amount of a composition 308 is positioned on anchoring layer 306. In some embodiments, anchoring layer 306 includes polydopamine, polylysine, fibronectin, laminin, collagen, or combinations thereof. In some embodiments, composition 308 is a hydrogel. In some embodiments, composition 308 includes a cell component 308C. In some embodiments, cell component 308C includes a concentration of cells. In some embodiments, cell component 308C includes cancer cells from a cancer cell line, primary cells from a tumor, or combinations thereof. In some embodiments, the cancer cells are from a known cancer cell line, e.g., MiaPaCa-2 pancreatic cancer cells, MCF7 and/or MDA-MB-231 breast cancer cells, etc. In some embodiments, the cancer cells are from a patient's own cancer cells. In some embodiments, the concentration of cells in composition 308 is between about 1×10⁵ cells/mL and about 1×10⁹ cells/mL composition. In some embodiments, the concentration of cells in composition 308 is between about 1×10⁷ cells/mL. In some embodiments, the amount of composition 308 is less than about 1 μL. In some embodiments, the amount of composition 308 is between about 60 nL and about 500 nL. In some embodiments, composition 308 includes a matrix component 308M. In some embodiments, matrix 308M includes Matrigel, alginate, collagen, peptides, or combinations thereof. In some embodiments, composition 308 includes a matrix component 308M and a cell component 308C. In some embodiments, cell component 308C is attached to a surface of composition 308 composed of matrix 308M. In some embodiments, composition 308 does not include matrix component 308M. In some embodiments, composition 308 includes one or more extracellular matrix proteins. In some embodiments, the extracellular matrix proteins include fibronectin, laminin, or combinations thereof. In some embodiments, composition 308 forms a generally planar layer (not shown). In some embodiments, composition 308 forms a generally spherical and/or hemi-spherical mass.

In an exemplary embodiment, a high throughput 330 micropillar-microwell sandwich platform was developed that enabled 3D co-culture of NK92-CD16 cells with pancreatic (MiaPaCa-2) and breast cancer cell lines (MCF7 and MDA-MB-231). The platform successfully mimicked hypoxic conditions found in a tumor microenvironment and was used to demonstrate NK-cell mediated cell cytotoxicity in combination with two monoclonal antibodies; Trastuzumab and Atezolizumab. The platform was also used to show dose response behavior of target cancer cells with reduced EC₅₀ values for paclitaxel (an anti-cancer chemotherapeutic) when treated with both NK cells and antibody. Such a platform can be used to develop more personalized cancer therapies using patient-derived cancer cells.

### EXAMPLES

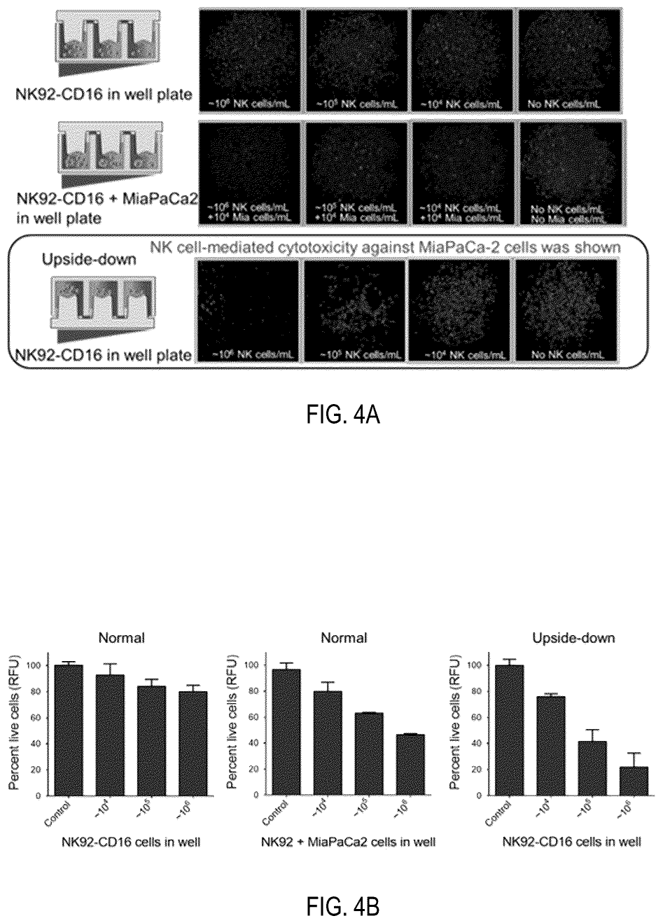

2D cancer cell culture for ADCC using 384-pillar/well sandwich platform. In an exemplary embodiment, ADCC of MiaPaCa-2 (pancreatic ductal adenocarcinoma cell line) was investigated initially using 2D cell culture within a 384-pillar/well sandwich platform, which includes a conventional 384-well plate along with a complementary plate with projecting pillars. The incubation of MiaPaCa-2 cells with NK92-CD16 was performed both upside down and normally. Normal orientation refers to incubating pillar surfaces face down on to the corresponding well plate while upside down orientation refers to incubating pillar surfaces face up on to the corresponding well plate. Direct contact between effector cells and target cells enabled efficient killing of MiaPaCa-2 cells. Specifically, as can be seen in FIGS. 4A and 4B, the percent of live cells were reduced to ˜20% when cells when pillars were incubated face up than with pillars face down. In addition, the presence of MiaPaCa-2 cells along with NK92-CD16 cells in the wells increased cell cytotoxicity against MiaPaCa-2 spotted on the pillars. This is likely due to granzyme release from interaction of NK92-CD16 cells and MiaPaCa-2 cells in the well plate. Without wishing to be bound by theory, previous work has shown that direct contact between effector and target cells upregulate expression of granzyme B. Because upside-down orientation of the pillar plate produced the most pronounced cancer cell killing, ADCC experiments were performed in this manner after addition of NK92-CD16 cells.

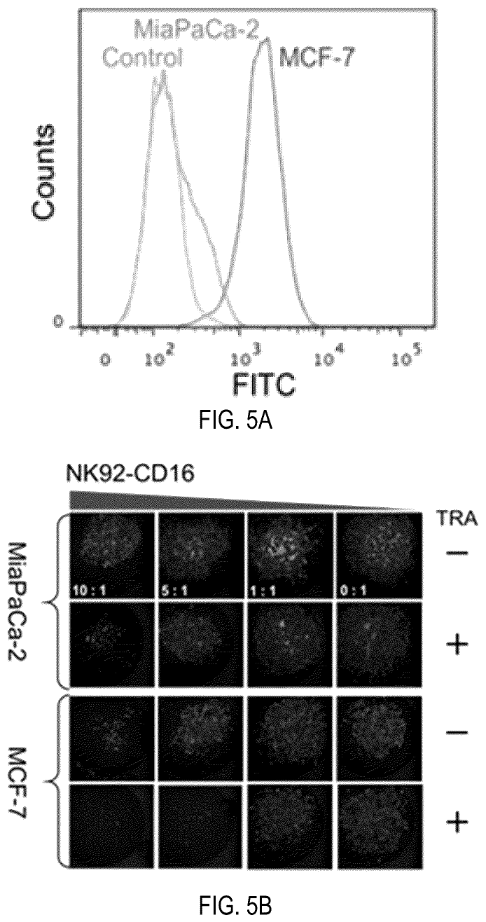

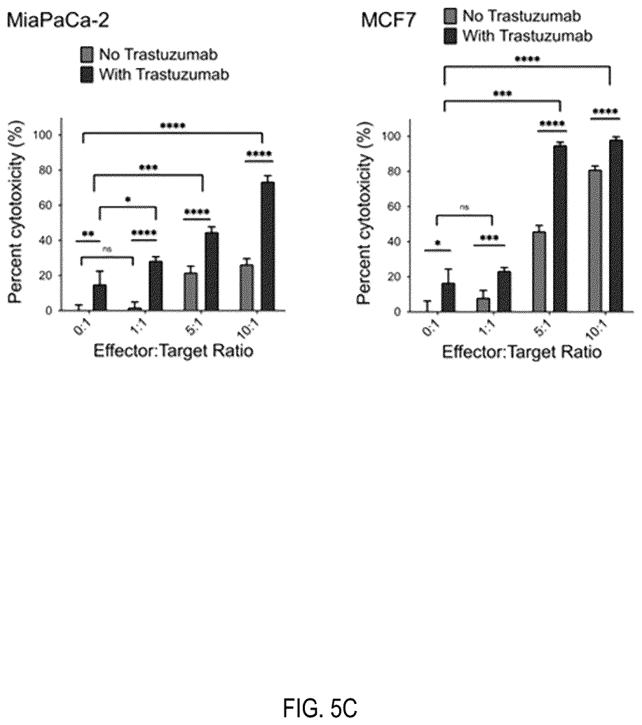

The 2D platform was used to investigate the effect of NK92-CD16 cell-mediated killing of both MiaPaCa-2 and a breast cancer cell line, MCF7, in the presence and absence of 5 μg/mL of Trastuzumab, which targets the HER2 receptor and has been used in breast cancer treatment. The cells were incubated with the antibody and NK cells for 24 h. The expression of HER2 was first evaluated on both MiaPaCa-2 and MCF7 cell lines using Alexa Fluor 488 conjugated Trastuzumab via flow cytometry (see FIG. 5A), which, consistent with the literature, indicated that MCF7 cells expressed more HER2 than MiaPaCa-2 cells. The effect of ADCC was then evaluated using NK92-CD16 on MiaPaCa-2 and MCF7 cells with and without Trastuzumab at three different effector to target (E:T) ratios. Both MCF7 and MiaPaCa-2 cells were cultured on 384-pillar/well sandwich platform as described previously. The percent cytotoxicity was calculated using ImageJ by measuring killing using NK92-CD16 cells vs. killing using saponin. The addition of Trastuzumab along with NK92-CD16 cells significantly enhanced killing efficiency against both cancer cell lines (see FIGS. 5B and 5C). Specifically, MCF7 cells exhibited higher cytotoxicity than MiaPaCa-2 cells at the same E:T ratio in the presence of Trastuzumab with close to 90% killing at an E:T ratio of 5:1 for MCF7 cells. This was expected given the higher expression of HER2 in MCF7 cells than in MiaPaCa-2 cells based on flow cytometry (see FIG. 5A). In addition, since NK92-CD16 cells were modified to express the CD16 receptor, the cells likely have the ability to bind to the Fc portion of Trastuzumab, thereby enabling greater killing efficiency.

These experiments show the feasibility of conducting ADCC with parallel exposure of effector cells and antibodies on the 384-pillar plate platform using conventional 2D cell culture methods. Several advantages in using this system for studying ADCC were evident, including easy removal of effector cells during the staining step as well as the absence of spontaneous release of Calcein-AM, since the cells do not require prestaining prior to treatment with NK92-CD16 cells. In addition, this 2D platform is compatible with high content imaging platforms for rapid testing of myriad therapeutic combinations.

3D cancer cell culture for NK-mediated cytotoxicity using 384-pillar/well sandwich platform. To enable 3D spheroid cancer cell culture on 384-pillar/well sandwich platform, the pillar surfaces were first coated with dopamine hydrochloride in Tris HCl to generate a polydopamine coating. MCF7 cells were then mixed with high concentration growth factor-reduced Matrigel (Corning Life Sciences, Corning, NY) and spotted on the coated pillar surface. While encapsulated cells grew as discrete aggregates for 5 days, the hanging drop method with Matrigel-cell mixture was unable to generate a single uniform spheroid. At the end of 5 days, the cells were exposed to NK92-CD16 cells overnight, then stained with Calcein-AM (to label live cells) and Hoechst 33342 (to label nuclei), and imaged using confocal microscopy.

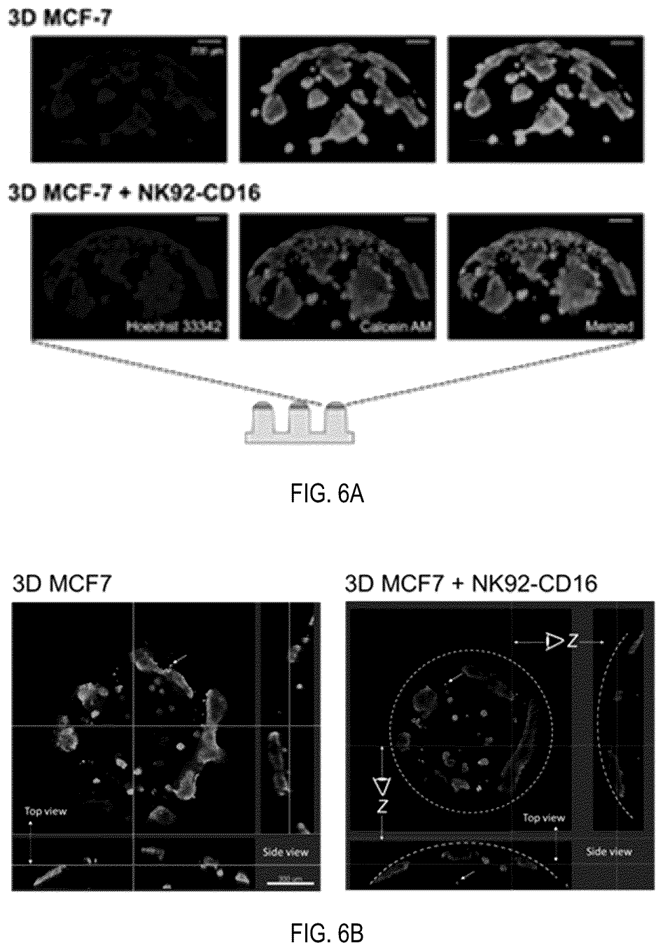

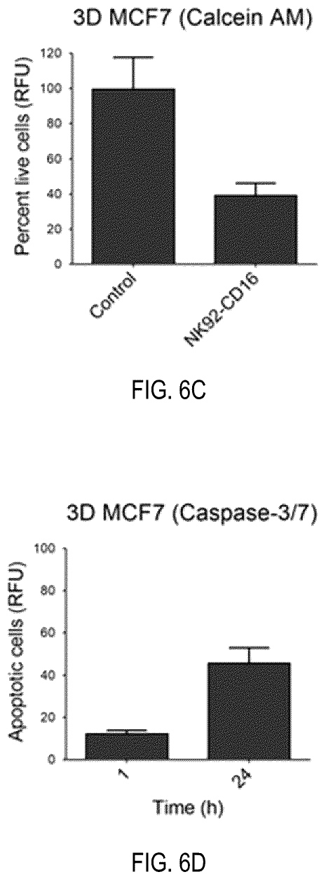

The effect of NK92-CD16 exposure on MCF7 aggregates in the 3D microenvironment on the pillar spot is shown in FIGS. 6A and 6B. There was a ˜60% reduction in the Calcein-AM staining for MCF7 aggregates in the presence of NK92-CD16 cells (see FIG. 6C). This indicates that NK92-CD16 cells can induce cytotoxicity of MCF7 aggregates even when the target cells are encapsulated in a 3D matrix. To demonstrate the migration of NK92-CD16 cells into Matrigel spots, triple color staining was carried out to label NK92-CD16 cells and MCF7 cells separately, with the NK cells labeled with CellTracker Deep Red prior to their addition to target MCF7 cells. MCF7 cells were contacted with NK92-CD16 cells for 1 h and 24 h. At the end of the exposure, both NK92-CD16 and MCF7 cells were stained with Caspase 3/7 Green to label dead cells and Hoechst 33342 to label all nuclei. The stained aggregates were then imaged using confocal microscopy. Exposure of MCF7 aggregates for 24 h showed increased Caspase-3/7 Green staining in comparison to 1 h exposure. Specifically, the percent apoptotic cells increased from ˜10% to ˜50% over 24 h (see FIG. 6D). This indicates that the MCF7 cells generate increased levels of Caspase 3 and Caspase 7 and become apoptotic due to the presence of NK92-CD16 cells. In addition, the presence of red staining indicated that NK92-CD16 cells migrated to the interior of the spot to kill MCF7 cells in the aggregates. Previous work has shown that NK92 cells express high levels of Matrix Metalloproteinases (MMPs) that allow for the local degradation of Matrigel proteins. If the MMPs in NK92 are able to cause disintegration of Matrigel, then they should be able to migrate inside the spot and target cancer cells that may be encapsulated in the matrix. The down-regulation of MMP expression has previously been shown to reduce Matrigel degradation using a cell invasion assay. These experiments established that NK92-CD16 cell-mediated cytotoxicity can be performed using the 384-pillar/well sandwich platform.

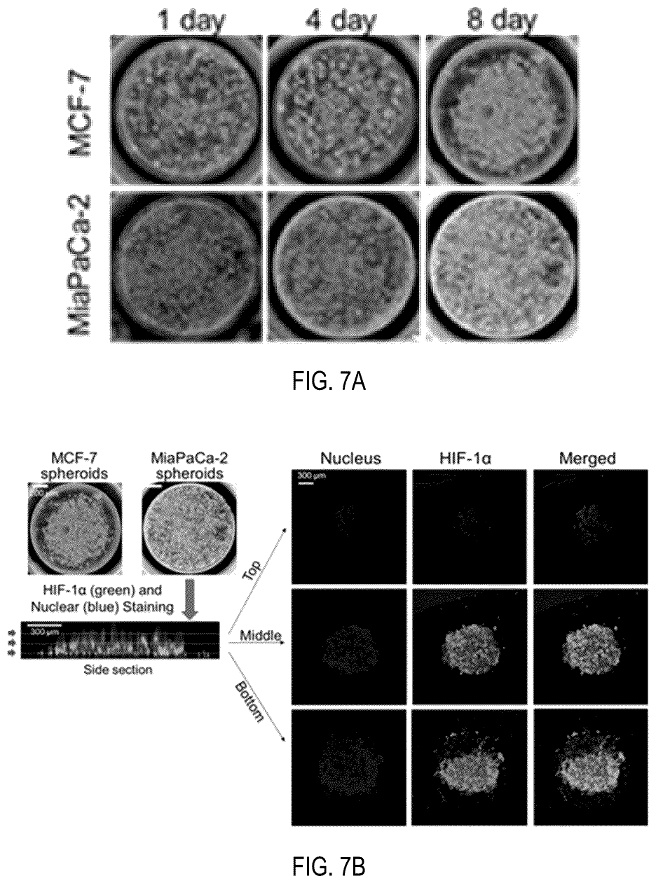

Uniform sized 3D tumor spheroid micropillar array for NK-mediated cytotoxicity. A 330 micropillar/microwell platform was used to generate target cell spheroids. This is similar to the 384-pillar/well sandwich platform described previously, albeit smaller in pillar surface area and overall size of the platform. The 330-micropillar and 330-microwell chips are approximately the size of a glass slide and the diameter of each pillar and well is 1 mm and 1.9 mm, respectively. Due to the reduced surface area, smaller volumes (250 nL) of Matrigel-cell mixtures at a high cell concentration (1×10⁷ cells/mL) were spotted on the surface of the micropillars. It was hypothesized that at high cell densities, as the target cells divide on the chip, multiple mini aggregates can combine to form a larger spheroid. The micropillar chips were incubated at 37° C. in a humidity chamber for 15 min following cell spotting to cause gelation of the spots and were then stamped with the corresponding microwell chip including 3.6 μL of medium per microwell. The sandwiched system was incubated in a humidity chamber to prevent evaporation of medium for the duration of culture of up to 8 days. The relatively high density of cells of 1×10⁷ cells/mL on the pillar surface (see FIG. 7A) enabled formation of spheroids as the cells grew within the Matrigel matrix on the pillar surface over the 8-day period.

To ensure that the TME was recapitulated, expression of HIF1α was measured using confocal microscopy. A HIF1α antibody was used to stain MiaPaCa-2 spheroids encapsulated in Matrigel after they were cultured for 5 days. A nuclear stain (Hoechst 33342) was used to label all cells within the spot. MiaPaCa-2 spheroids stained positively for HIF1α indicating that the spheroids exhibited hypoxia (FIG. 7B). A cross-sectional view of the spheroids reveals that HIF1α is more highly expressed at the bottom and middle of the spheroid than at the top. This is to be expected as both the bottom and middle of the spheroid have reduced access to oxygen in comparison to the cells at the top, which maintains direct contact with air. These results indicate that high density culture of target cancer cells can accurately represent the hypoxic conditions in a TME.

High-content imaging of 3D tumor spheroid micropillar array for effector cell-mediated cytotoxicity including NK ADCC/drug combination. Uniform-sized 3D spheroids in the 330-micropillar array were used to perform high-throughput ADCC on three different cancer cell lines; MiaPaCa-2, MCF7 and MDA-MB-231, the latter a triple negative breast cancer cell line, in the presence and absence of two chemotherapeutic drugs, doxorubicin and paclitaxel. The ADCC screen was performed by spotting 250 nL onto polydopamine-coated micropillars including 2500 target cancer cells per spot. The cells were cultured for 3 days and then treated with NK92-CD16 cells. Three different E:T ratios (1:1, 5:1 and 10:1) were tested in the presence and absence of 2.5 μg/mL Trastuzumab, 3.5 nM doxorubicin or 7.0 nM paclitaxel. The antibody concentration was chosen based on previous ADCC experiments in 2D pillar/well sandwich platform and the drug concentrations were chosen to be approximately 10-fold lower than the EC50 values obtained from 2D cell culture for the three cell lines. Combinatorial addition of both Trastuzumab and doxorubicin, as well as Trastuzumab and paclitaxel was also performed at all three E:T ratios. As a control, one block of spots was reserved for exposure of target cells with both antibody and the drugs but without NK92-CD16 cells. The micropillar/microwell sandwich platform was incubated for 24 h upside-down to allow for target cell killing on culture day 3. The next day, the chips were then washed, stained with Hoechst 33342, Calcein-AM and Propidium Iodide, and imaged using the ASFA™ scanner.

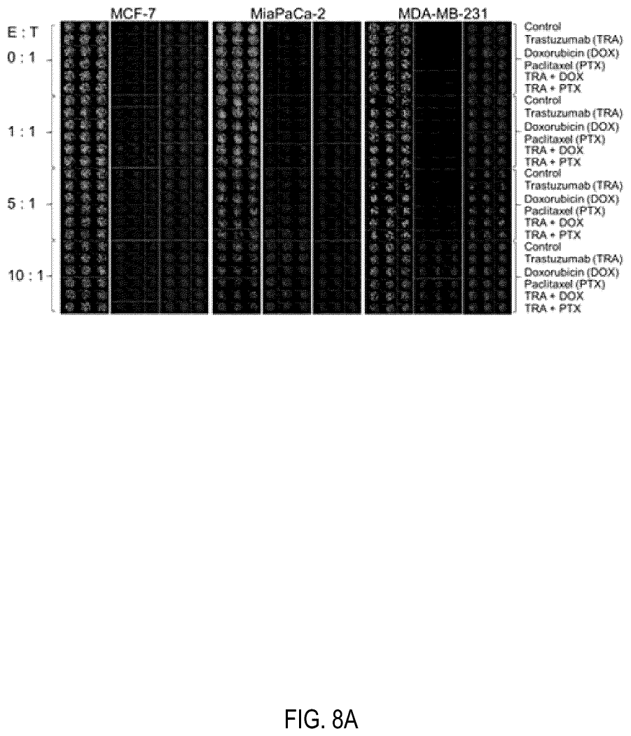

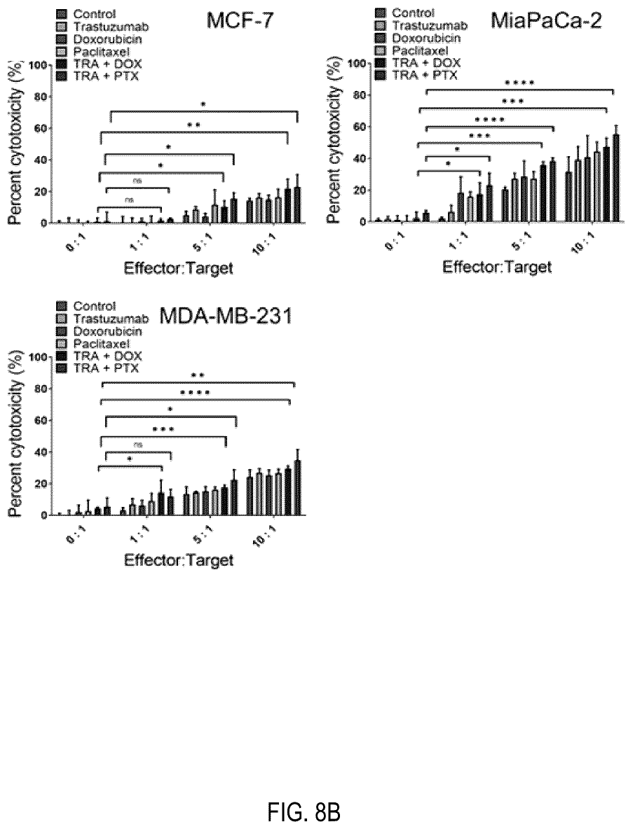

Referring now to FIGS. 8A and 8B, for all three cell lines, increasing the E:T ratio from 1:1 to 10:1 increased target cell cytotoxicity. As the number of NK92-CD16 cells added was increased, they were more effective in killing target cancer cell spheroids. The combination of NK92-CD16 at the E:T ratio of 10:1, Trastuzumab and doxorubicin or paclitaxel was significantly more effective in target cell cytotoxicity (p<0.01 or <0.05 for doxorubicin or paclitaxel, respectively) for all three cell lines. In addition, there were minimal differences in cytotoxic responses of the three cancer cell lines among the antibody only, drug only, and combined antibody-drug conditions at all three E:T ratios when the cancer cells were incubated for 24 h. This indicates that the primary contributor for cell death in target cells is the presence of NK92-CD16 cells. In addition, among the three cell lines tested, MiaPaCa-2 spheroids exhibited the greatest cytotoxicity in response to NK92-CD16 exposure, followed by MDA-MB-231 and then MCF7 spheroids. This is contrary to what was observed in 2D monolayer culture where MCF7 cells were found to be more likely to die upon NK92-CD16 exposure than MiaPaCa-2 cells. This may be due to acquired resistance of MCF7 cells when they were cultured in 3D spheroids. Previous work has shown that the expression of HIF11α in MCF7 contributes to its resistance to chemotherapeutic drugs including doxorubicin. In addition, when certain breast cancer cells were cultured under hypoxic conditions in 3D, they were found to acquire resistance to Trastuzumab through changes in the HER2 expression causing a diminished response to the antibody. Furthermore, MiaPaCa-2 cells display higher basal levels of MMPs than MCF7. The presence of MMPs in cells can cause them to become highly metastatic and invasive. Indeed, MiaPaCa-2 cells have the ability to penetrate Matrigel using Transwell Invasion assay. This implies that a similar process may occur in the micropillar-microwell systems of the present disclosure, wherein the degradation of the Matrigel surrounding the target cells may provide easier access for NK92-CD16 cells to infiltrate the 3D structure. This increased access produces higher cytotoxicity against MiaPaCa-2 cells than against MCF7 cells that do not express high basal levels of MMPs. Similarly, MDA-MB-231, a highly invasive breast cancer cell line, has high expression of MMP9. This could explain the observed higher cytotoxicity behavior in MDA-MB-231 spheroids in comparison to MCF7 spheroids.

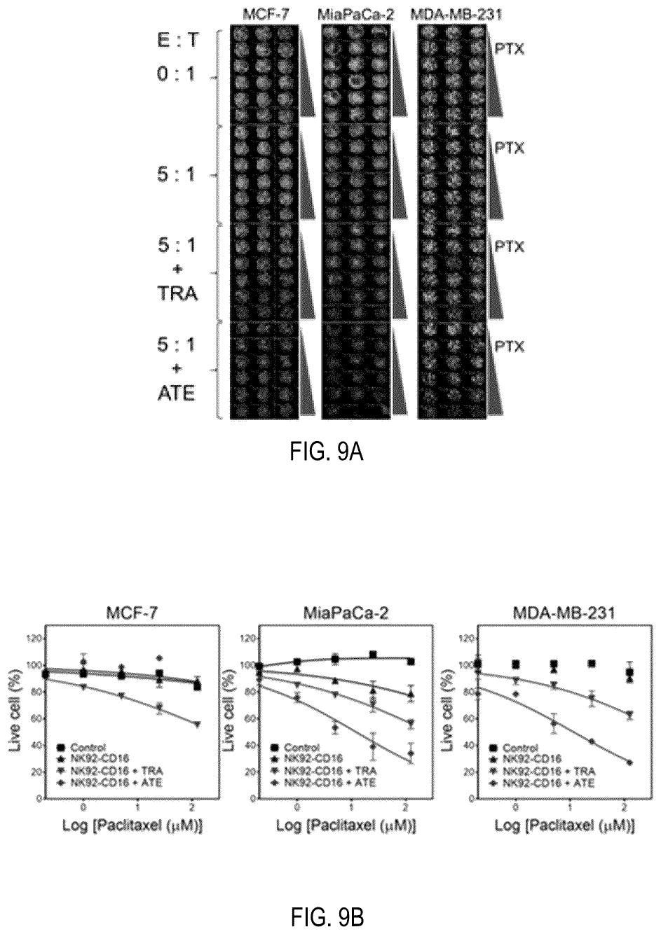

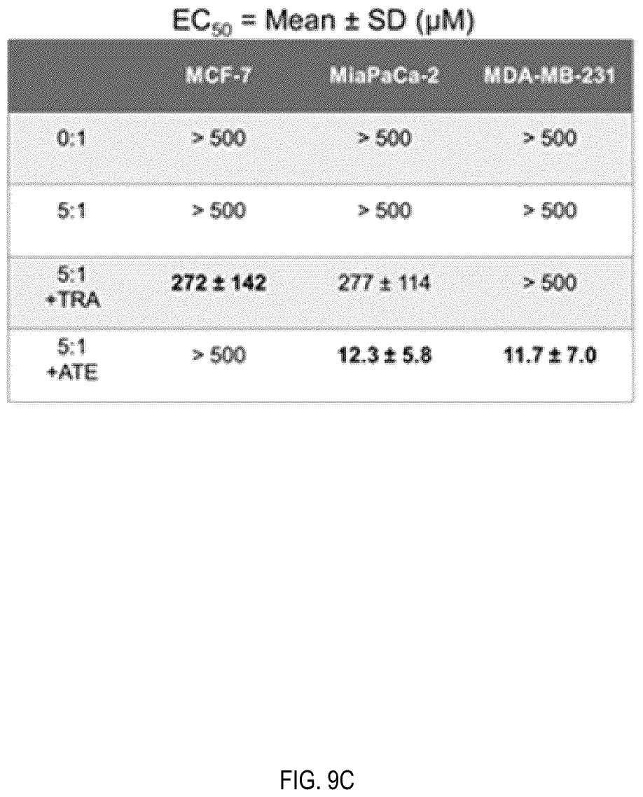

Since all three cell lines displayed increased cell death upon combinatorial exposure of NK92-CD16 with doxorubicin or paclitaxel and Trastuzumab, a second antibody was tested, Atezolizumab, an anti-Programmed Death Ligand 1 (PDL1) antibody that acts as a checkpoint inhibitor. Dose response behavior of the three target cell lines was observed upon exposure to Atezolizumab plus paclitaxel at the same NK92-CD16 ratio (see FIGS. 9A-9C). There was interest in whether MDA-MB-231, which is a triple negative breast cancer cell line having reduced expression of estrogen receptor (ER), progesterone receptor and HER252, was susceptible to cell death upon combination exposure of paclitaxel, Atezolizumab and NK92-CD16 cells. Triple negative breast cancers are particularly aggressive to treat due to very low expression levels of ER, PR and HER253. To test this, the three target cancer cell lines were spotted onto the 330-micropillar platform to generate the corresponding 3D spheroids. A range of paclitaxel concentrations were used in the presence and absence of NK92-CD16, Trastuzumab and Atezolizumab. The 3D spheroids on the micropillar chip were incubated for 24 h, and then stained and imaged. The killing of MCF7 tumor spheroids was augmented by the presence of the Trastuzumab and NK92-CD16 cells, presumably due to HER2 antibody-dependent NK cell-mediated cytotoxicity (see FIGS. 9A-9C). In the absence of the effector with NK92-CD16 cells, minimal paclitaxel-induced cytotoxicity was observed (see FIG. 9C) with EC50 values >500 μM. For MCF7, the most significantly cytotoxic condition was obtained with paclitaxel in the presence of both Trastuzumab and NK92-CD16 cells; approximately 1.8-fold lower EC50 value than in the absence of the antibody and effector cells. Interestingly, the same condition against MDA-MB-231 spheroids did not prove to be effective. Rather, an approximately 43-fold lower EC50 value was obtained for paclitaxel in the presence of Atezolizumab along with NK92-CD16 cells. MDA-MB-231 cells express high levels of PDL-154, and hence, an antibody targeting this receptor should be able to augment the killing of MDA-MB-231 cells in the presence of paclitaxel. For MiaPaCa-2 spheroids, both antibodies were effective in inducing cytotoxicity with Atezolizumab performing substantially better than Trastuzumab.

### Methods

Cancer cell and NK92-CD16 culture. MiaPaCa-2 cells were cultured in Dulbecco's Modified Eagle Medium (DMEM) supplemented with 10% Fetal Bovine Serum (FBS) and 1% Penicillin-Streptomycin (Pen-Strep). MCF7 cells were cultured in DMEM/F12 supplemented with 10% FBS, 0.01 mg/mL insulin and 2 mM Glutamax and 1% Pen-Strep. MDA-MB-231 cells were cultured in DMEM (high glucose) supplemented with 10% FBS and 1% Pen-Strep. NK92-CD16 cells were cultured in Prime XV NK Cell Chemically Defined Medium (Irvine Scientific, Irvine, CA) supplemented with 1000 IU/mL of IL-2 (PeproTech, Cranbury, NJ) and 1% Pen-Strep. The cancer cells were grown in T75 flasks and NK cells were grown in 24-well and 6-well ultra-low attachment plates. The cells were all maintained in a 37° C. incubator at 5% CO₂. The cancer cells were passaged when they reached 70-80% confluent. The NK cells were passaged when they reached 1×10⁶ cells/mL and medium was exchanged every 2 days.

2D and 3D experiments on a 384-pillar/well sandwich system. Polystyrene 384 pillar plates (MBD Korea Co., South Korea) were used for both 2D and 3D cell culture experiments. For 2D experiments, the pillar plates were coated with 1% fibronectin from bovine plasma by stamping the pillar plates on 384 well plates including 40 μL of 1% fibronectin in DPBS. The sandwiched plates were placed in 37° C. for 1 h. The pillar plates were then removed from the solution and stored at 4° C. until they were ready for printing. For cell printing, target cancer cells were washed with Dulbecco's Phosphate Buffered Saline (DPBS), detached with 0.25% Trypsin-EDTA and spun down at 150 g for 5 min. The cells were resuspended in fresh medium at a concentration of 2.5×10⁶ cells/mL. Cell suspension (1 μL) was spotted on fibronectin coated plates using an ASFA™ fifth generation cell spotter (MBD Korea Co., South Korea). The plate was then incubated in a humidity chamber with pillars facing up at 37° C. for 6 h to allow for cell attachment. The pillar plate was then sandwiched with a conventional 384-well plate including fresh medium. After 2-3 days, NK92-CD16 cells were added with or without 1 μg/mL and 5 μg/mL Trastuzumab in 384-well plates. The pillar plates including target cells were stamped with the well plates including NK cells and incubated upside down overnight. The next day the pillar plates were stained in 4 μM Calcein-AM and 5 μg/mL Hoechst 33342 diluted in fresh medium for 30 minutes at 37° C. The plate was then imaged using a Cellomics ArrayScan VTI (Thermo Fisher Scientific, Waltham, MA). Image analysis for quantifying fluorescent intensities and merging images were performed using ImageJ software (NIH).

For 3D cell culture, 384 pillar plates were first coated with polydopamine by incubating in a solution including 2 mg/mL dopamine hydrochloride in Tris HCl at pH 8.5 for 2 h on a room temperature shaking incubator at 120 rpm. The plates were washed with DI water, dried and stored until further use. The cancer cells were harvested as described previously and mixed with high concentration growth factor reduced Matrigel (Basement membrane extracted from Engelbreth-Holm-Swarm mouse sarcoma, Corning Life Sciences Catalog #354263) to a final cell concentration of 2.5×10⁶ cells/mL for pillar plate. Matrigel-cell mixture (1 μL) was then spotted using the precooled ASFA™ spotter onto coated pillar plates, which were then incubated face down at 4° C. for 15 min to allow for cell aggregation at the bottom of the spots. The plates were then placed at 37° C. in a humidity chamber to allow for spot gelation. The pillar plates were subsequently sandwiched with well plates including fresh medium.

3D experiments on 330-micropillar/microwell sandwich system. Polystyrene 330 micropillar chips (MBD Korea Co., South Korea) were coated with polydopamine, washed and dried until further use. The 330 well chips were UV-treated using a 96 W transilluminator (Syngene GVM-30) for 4 h to enhance surface hydrophilicity. Cancer cells were detached as described previously and mixed with high concentration growth factor-reduced Matrigel to obtain a final cell concentration of 1×10⁷ cells/mL. Then, 250 nL of the Matrigel-cell mixture was spotted using the ASFA™ spotter onto the polydopamine coated 330 micropillar chips. The chips were incubated face down in a humidity chamber at 37° C. for 15 min to allow for gelation and then they were sandwiched with the corresponding 330 microwell chip including 3.6 μL of fresh medium per well. The chips were cultured for a period of 3 days prior to NK cell exposure. The cell growth medium on the chip was exchanged every 2 days. For cytotoxicity experiments, where the NK92-CD16 ratio was varied, 25 different conditions were tested (see Table 1 below), including a dead control (via addition of saponin).

TABLE 1
Conditions tested for cytotoxicity experiments.

| | Effector: | |
|---|---|---|
| | Target | |
| Condition | Ratio | Drugs/Antibody |
| 1 | No NK | No Drug/Antibody |
| 2 | No NK | 2.5 μg/mL of Trastuzumab |
| 3 | No NK | 3.5 nM Doxorubicin |
| 4 | No NK | 7.0 nM Paclitaxel |
| 5 | No NK | 2.5 μg/mL of Trastuzumab + 3.5 nM Doxorubicin |
| 6 | No NK | 0.5 μg/mL of Trastuzumab + 7.0 nM Paclitaxel |
| 7 | 1:1 | No Drug/Antibody |
| 8 | 1:1 | 2.5 μg/mL of Trastuzumab |
| 9 | 1:1 | 3.5 nM Doxorubicin |
| 10 | 1:1 | 7.0 nM Paclitaxel |
| 11 | 1:1 | 2.5 μg/mL of Trastuzumab + 3.5 nM Doxorubicin |
| 12 | 1:1 | 0.5 μg/mL of Trastuzumab + 7.0 nM Paclitaxel |
| 13 | 5:1 | No Drug/Antibody |
| 14 | 5:1 | 2.5 μg/mL of Trastuzumab |
| 15 | 5:1 | 3.5 nM Doxorubicin |
| 16 | 5:1 | 7.0 nM Paclitaxel |
| 17 | 5:1 | 2.5 μg/mL of Trastuzumab + 3.5 nM Doxorubicin |
| 18 | 5:1 | 0.5 μg/mL of Trastuzumab + 7.0 nM Paclitaxel |
| 19 | 10:1 | No Drug/Antibody |
| 20 | 10:1 | 2.5 μg/mL of Trastuzumab |
| 21 | 10:1 | 0.0035 μM Doxorubicin |
| 22 | 10:1 | 7.0 nM Paclitaxel |
| 23 | 10:1 | 2.5 g/mL of Trastuzumab + 3.5 nM Doxorubicin |
| 24 | 10:1 | 0.5 μg/mL of Trastuzumab + 7.0 nM Paclitaxel |
| 25 | No NK | Dead Control (Saponin Treated) |

Similarly, for the dose response experiments with paclitaxel, where the E:T ratio was fixed at 5:1, 25 different conditions including a dead cell control were evaluated, as shown in Table 2.

TABLE 2
Conditions tested for dose response experiments.

| Condition | NK Cells | Paclitaxel Concentration | |
|---|---|---|---|
| 1 | No NK | 0 | μM |
| 2 | No NK | 0.02 | μM |
| 3 | No NK | 1 | μM |
| 4 | No NK | 5 | μM |
| 5 | No NK | 25 | μM |
| 6 | No NK | 125 | μM |
| 7 | 5:1 | 0 | μM |
| 8 | 5:1 | 0.02 | μM |
| 9 | 5:1 | 1 | μM |
| 10 | 5:1 | 5 | μM |
| 11 | 5:1 | 25 | μM |
| 12 | 5:1 | 125 | μM |
| 13 | 5:1 + Trastuzumab (2.5 μg/mL) | 0 | μM |
| 14 | 5:1 + Trastuzumab (2.5 μg/mL) | 0.02 | μM |
| 15 | 5:1 + Trastuzumab (2.5 μg/mL) | 1 | μM |
| 16 | 5:1 + Trastuzumab (2.5 μg/mL) | 5 | μM |
| 17 | 5:1 + Trastuzumab (2.5 μg/mL) | 25 | μM |
| 18 | 5:1 + Trastuzumab (2.5 μg/mL) | 125 | μM |
| 19 | 5:1 + Atezolizumab(2.5 μg/mL) | 0 | μM |
| 20 | 5:1 + Atezolizumab(2.5 μg/mL) | 0.02 | μM |
| 21 | 5:1 + Atezolizumab(2.5 μg/mL) | 1 | μM |
| 22 | 5:1 + Atezolizumab(2.5 μg/mL) | 5 | μM |
| 23 | 5:1 + Atezolizumab(2.5 μg/mL) | 25 | μM |
| 24 | 5:1 + Atezolizumab(2.5 μg/mL) | 125 | μM |

| | | |
|---|---|---|
| 25 | No NK | Dead Control (Saponin) |

Unconjugated anti-HER2 antibody was used for experiments on 330 micropillar/microwell chips. The NK92-CD16 cells, Trastuzumab, and the drugs were spotted on 330 microwell chip. The micropillar chip with target cells was then stamped onto the 330 microwell chip and incubated upside down for 24 h. The micropillar chips were then washed with 8 mL DPBS added to a 4-well rectangular dish and stained with 4 μM Calcein AM (for live-cell staining), 10 μg/mL Propidium Iodide (for dead-cell staining) and 5 μg/mL Hoechst 33342 (for nuclear staining) in 8 mL of DPBS with 1 g/L, D-glucose for 30 min. The chips were subsequently washed in DPBS and imaged using an ASFA™ Cell Scanner (MBD Korea Co., South Korea) and quantitatively analyzed using cell analyzer software within the imaging system.

Immunofluorescence. Immunofluorescence was performed on MiaPaCa-2 3D spheroids to measure expression of Hypoxia Inducing Factor 1α (HIF11α). To generate spheroids, 250 nL of Matrigel-cell mixture (at a concentration of 1×10⁷ cells/mL) was spotted on a glass bottom confocal dish using the ASFA™ spotter. The spots were gelled by incubating the dish upside down in a humidity chamber for 15 min at 37° C. Fresh DMEM was added to the confocal dish to cover the spots and the spheroids were cultured for 5 days, and then the spheroids were washed with DPBS and fixed with 4% (w/v) paraformaldehyde and 0.25% glutaraldehyde (w/v) in DPBS for 20 min. The spheroids were permeabilized with 0.25% (v/v) Triton X-100 in DPBS for 30 min and quenched with 2 mg/mL sodium borohydride in DPBS. The spheroids were then blocked with 5% (w/v) bovine serum albumin and 1% (v/v) goat serum in DPBS overnight at 4° C. and stained with mouse 10 μg/mL anti-HIF1α antibody (R&D systems MAB1536) in 1% (w/v) BSA in DPBS overnight at 4° C. An Alexa Fluor 488 goat anti-mouse (Invitrogen A28175) antibody was used as a secondary antibody with staining done in 1% BSA in DPBS overnight at 4° C. at 1:500 dilution. The cells were then stained with 5 μg/mL Hoechst 33342 for 10 min at room temperature and imaged using a Leica TCS SP8 STED Confocal Microscope. Z-stacks of the spheroids were constructed using Imaris Viewer.

Cell viability and apoptosis. Confocal microscopy was performed on MCF7 aggregates in 3D to evaluate NK92-CD16 cytotoxicity with Calcein AM staining and triple color staining. For Calcein-AM staining, MCF7 cells were mixed with high concentration growth factor-reduced Matrigel at a final concentration of 2.5×10⁶ cells/mL and spotted onto a glass bottom confocal dish. The 3D aggregates were cultured for 5 days and then stained with 4 μM Calcein-AM and 5 μg/mL Hoechst 33342 diluted in DPBS with 1 g/L D-glucose for 30 min at 37° C. For triple color staining with Cell Event Caspase 3/7 Green, Cell Tracker Deep Red and Hoechst, MCF7 cells were spotted on glass bottom confocal dish as before. NK92-CD16 cells were pre-stained with 1 μM Cell Tracker Deep Red prior to their addition to MCF7 aggregates. The stained NK92-CD16 cells were added to the MCF7 aggregates and incubated for 1 h and 24 h. The MCF7 aggregates were then stained with 4 μM Cell Event Caspase 3/7 Green and 5 μg/mL Hoechst 33342. The aggregates were subsequently imaged using Leica TCS SP8 STED Confocal Microscope. Z-stacks of the spheroids were constructed using Imaris Viewer.

Flow cytometry. For detection of HER-2 expression using flow cytometry, 1×10⁶ MCF7 and MiaPaCa-2 cells were detached using 0.25% Trypsin EDTA and washed with DPBS. The cells were then stained with 10 μg/mL of Alexa Fluor 488 conjugated Anti-iER2 antibody diluted in 1% (w/v) BSA in DPBS. The cells were then washed 3-times in DPBS to remove any unbound antibody and then submitted to flow cytometry (BD LSR II). A negative control that was not stained was also run to establish the fluorescence gates. The results were then analyzed using Flow Jo v7.6.

Statistical analysis and EC₅₀ measurements. All results were analyzed using GraphPad Prism. The error bars on figures indicate mean±standard deviation (SD) unless otherwise indicated. An unpaired student's t-test was run for measuring statistical significance, and p<0.05 was considered significant. For dose-response curves without clearly defined top or bottom plateaus, these values were set to either the maximum or minimum viability values within the sampled range. The sigmoidal dose-response curves and EC₅₀ values for a drug compound were obtained using Eq. 1:

$$Y = Bottom + \frac{( Top - Bottom )}{1 + {10}^{( LogEC 50 - X ) eH}} \qquad ( 1 )$$

where EC₅₀ is the midpoint of the curve, H is the hill slope, X is the logarithm of a drug concentration, and Y is the response (% live cells), starting at Bottom and going to Top with a sigmoid shape. The log(EC₅₀) values were obtained from the dose response curves generated in GraphPad Prism.

The micropillar platform is effective in multiple designs for use in ADCC screening of NK-cell activity against tumor cell lines, e.g., human pancreatic ductal adenocarcinoma cell line (MiaPaCa-2) and human breast adenocarcinoma cell line (MCF7). Specifically, a simple 2D cancer cell monolayer can be extended to a 3D model with the cells embedded in a matrix, e.g., Matrigel, and then to uniform-sized tumor spheroids. The latter provides conditions for screening in a contextually relevant 3D microenvironment. Differences in NK92-CD16 mediated cytotoxicity was evident between 2D and 3D environments. For example, while MCF7 cells exhibited high levels of cell death due to ADCC in 2D, the same cells were much more resistant to NK92-CD16 cells when they were cultured as 3D spheroids. The presence of an extracellular matrix can limit the cytotoxicity of drugs and antibodies, and can play a major role in determining the response of cancer to a particular treatment, and such response may be poorly predictive using in conventional 2D in vitro models. The micropillar-microwell platform, particularly under conditions that mimic the tumor microenvironment, therefore, has the potential to serve as a useful tool for early-stage immunotherapy discovery.

The resistance of cancer cells to several chemotherapeutic drugs may be overcome by coupling them to immunotherapeutics. Although paclitaxel alone was poorly effective as a cytotoxic agent against both MCF7 and MDA-MB-231 cells in spheroid cultures, the addition of NK-92 CD16 cells with Trastuzumab or Atezolizumab increased cytotoxicity. Indeed, combinatorial treatments have been shown to be more successful in clinical trials for breast cancer patients than drug or antibody treatment alone. These results could be attributed to the synergistic effects of drug, antibody and immune cells. Such synergism has been attributed to immune cell recognition and targeting of cancer cells upon release of stress associated factors, i.e., Heat Shock Proteins (HSPs), High-Mobility Group Box 1 Proteins (HMGBIs), etc., in the presence of chemotherapeutic drugs. In addition, reorganization of cell surface receptors upon addition of cytotoxic drugs is known to make cancer cells more vulnerable to immune cell mediated cell death. These results serve to demonstrate how combination of ADCC with chemotherapeutic drugs may impact solid tumors.

Methods and systems of the present disclosure are advantageous to provide a high throughput micropillar-microwell sandwich 3D cell culture platform to overcome the aforementioned limitations of current high-throughput methods. This platform enables the generation of 3D tumor spheroids on a micropillar surface to mimic the tumor microenvironment, as well as co-culture of cancer cells and NK cells for investigation of ADCC. Moreover, this platform facilitates rapid quantification of cytotoxicity without pre-labeling of target cells, separate labeling of effector and target cells with different fluorescent probes, or long processing times. As a result, several combinations of NK92 cells and antibodies have been identified that induce cytotoxicity in multiple metastatic cancer spheroids. The dose response behavior of cancer spheroids when they were exposed to a known a chemotherapeutic drug, paclitaxel, in combination with antibody treated NK92-CD16 cells was investigated. This platform can serve as an effective high-throughput, high-content screening tool for the development of personalized immunotherapies.

Therefore, the platform of the present disclosure appears to recapitulate the complex interactions that occur between immune cells and therapeutics within the TME. The presence of NK92-CD16 cells was able to accelerate the process of cytotoxicity to 24 h in combination with the drugs. This is particularly relevant as previous reports have shown that breast cancer cells can exhibit short-term resistance during drug exposure. Therefore, the platform may be used to screen and identify novel immunotherapy combinations to treat highly aggressive forms of cancers.

A 330-micropillar/microwell sandwich platform was developed that enables co-culture of both immune cells and various 3D cancer spheroids. The platform faithfully recapitulated the hypoxic environment that is often present within 3D tumor spheroids and that make target cells more resistant to certain therapeutics. This system was also able to use Fragment crystallizable Region (FcR)-mediated immune effector engagement with NK92-CD16 cells to induce ADCC against multiple different cancer cell lines. The 330-micropillar/microwell sandwich platform, therefore, may be useful in screening patient-derived cancer cells to develop more effective personalized therapies.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A method of performing cancer-immune cell co-culture, comprising:
applying an anchoring layer to a micropillar chip;
preparing a composition including a plurality of cancer cells;
spotting an amount of the composition on the anchoring layer of a plurality of micropillars on the micropillar chip; and
incubating the composition on the micropillar chip,
wherein the plurality of cancer cells is from a cancer cell line, primary cells from a tumor, or combinations thereof.

2. The method according to claim 1, further comprising:
applying a medium to microwells of a microwell chip, the medium including a cell culture including a concentration of immune cells;
immersing the plurality of micropillars in the medium on the microwell chip to facilitate co-culture of the cancer cells and the immune cells; and
identifying a cytotoxic effect of the medium on the cancer cells.

3. The method according to claim 1, wherein the composition is a hydrogel.

4. The method according to claim 3, wherein the composition includes a matrix, the matrix including Matrigel, alginate, collagen, peptides, or combinations thereof.

5. The method according to claim 4, wherein the composition further comprises one or more extracellular matrix proteins including fibronectin, laminin, or combinations thereof.

6. The method according to claim 1, wherein the anchoring layer includes polydopamine, polylysine, fibronectin, laminin, collagen, or combinations thereof.

7. The method according to claim 1, wherein the amount of the composition is less than about 1 μL.

8. The method according to claim 7, wherein the amount of composition is between about 60 nL and about 500 nL.

9. The method according to claim 1, wherein the composition has a cell concentration between about 1×10⁵ cells/mL and about 1×10⁹ cells/mL.

10. The method according to claim 2, wherein the immune cells include lymphocytes, monocytes, macrophages, peripheral blood mononuclear cells, dendritic cells, or combinations thereof.

11. The method according to claim 10, wherein the medium further includes one or more treatments, the treatments including a concentration of antibodies, a concentration of therapeutic small molecules, or combinations thereof.

12. A method of evaluating cancer cell cytotoxicity, comprising:
applying an anchoring layer to a plurality of micropillars formed on a micropillar chip;
preparing a composition including a matrix and a plurality of cancer cells;
spotting an amount of the composition on the anchoring layer on a plurality of the micropillars;
incubating the composition on the micropillar chip to form sphereoids of composition;
applying growth medium to a plurality of microwells of a microwell chip, wherein the plurality of micropillars are configured to fit within the plurality of microwells;
immersing the spheroids in the growth medium;
applying one or more treatments against the plurality of cancer cells to the microwells; and
identifying the effect of the one or more treatments on the plurality of cells, wherein the anchoring layer includes polydopamine, polylysine, fibronectin, laminin, collagen, or combinations thereof, and
wherein the plurality of cancer cells is from a cancer cell line, primary cells from a tumor, or combinations thereof.

13. The method according to claim 12, wherein the one or more treatments include a concentration of natural killer (NK) cells, a concentration of antibodies, a concentration of therapeutic small molecules, or combinations thereof.

14. The method according to claim 12, wherein the composition is a hydrogel.

15. The method according to claim 14, wherein the matrix includes Matrigel, alginate, collagen, peptides, or combinations thereof.

16. The method according to claim 15, wherein the composition further comprises one or more extracellular matrix proteins, the extracellular matrix proteins including fibronectin, laminin, or combinations thereof.

17. The method according to claim 12, wherein the amount of composition is between about 60 nL and about 500 nL.

18. The method according to claim 12, wherein the composition has a cell concentration between about 1×10⁵ cells/mL and about 1×10⁹ cells/mL.

19. A chip system comprising:
a micropillar chip and a microwell chip, the micropillar chip including a plurality of micropillars configured to fit within corresponding microwells on the microwell chip;
an anchoring layer on at least a portion of the micropillars;
an amount of a hydrogel composition on the anchoring layer, the hydrogel composition including:
a matrix including Matrigel, alginate, collagen, peptides, or combinations thereof, and
a concentration of cells between about 1×10⁵ cells/mL and about 1×10⁹ cells/mL composition,
wherein the amount of the hydrogel composition is between about 60 nL and about 500 nL,
wherein the anchoring layer includes polydopamine, polylysine, fibronectin, laminin, collagen, or combinations thereof, and
wherein the cells are from a cancer cell line, primary cells from a tumor, or combinations thereof.

20. The chip system according to claim 19, wherein the hydrogel composition further comprises one or more extracellular matrix proteins.
